(19) United States
(12) Patent Application Publication
(10) Pub. No.: US 2010/0305635 A1
(43) Pub. Date: Dec. 2, 2010

(54) SYSTEM AND METHOD FOR RHYTHM IDENTIFICATION AND THERAPY DISCRIMINATION USING HEMODYNAMIC STATUS INFORMATION

(76) Inventors: Lili Liu, Maple Grove, MN (US); Dan Li, Shoreview, MN (US); Allan C. Shuros, St. Paul, MN (US); James O. Gilkerson, Stillwater, MN (US)

Correspondence Address: FAEGRE & BENSON LLP;PATENT DOCKETING - INTELLECTUAL PROPERTY (32469), 2200 WELLS FARGO CENTER, 90 SOUTH SEVENTH STREET, MINNEAPOLIS, MN, 55402-3901, US

(21) Appl. No.: 12/786,074

(22) Filed: May 24, 2010

Related U.S. Application Data
(60) Provisional application No. 61/181,186, filed on May 26, 2009.

(51) Int. Cl. A61N 1/365; A61N 1/39

(52) U.S. Cl. 607/6; 607/14

(57) ABSTRACT

A system and method for controlling cardiac ventricular tachyarrhythmias by acquiring a pressure signal representative of coronary venous pressure (CVP) from a pressure sensor implanted within a coronary vein of the patient. A CVP index is derived based on the pressure signal. The onset of a ventricular tachyarrhythmia episode is detected based on a cardiac rates signal. The CVP index and the rate signal are monitored and, responsive to the rate signal indicating a sustained tachycardia episode during the episode monitoring period, anti-tachycardia therapy selectively withheld and the episode monitoring period is extended based on the CVP index.

### CROSS-REFERENCE TO RELATED APPLICATIONS

This application claims the benefit of U.S. Provisional Application No. 61/181,186, filed May 26, 2009, entitled “System and Method for Rhythm Identification and Therapy Discrimination using Hemodynamic Status Information,” which is incorporated herein by reference in its entirety.

### TECHNICAL FIELD

The present invention relates generally to implantable pulse generators and in particular to implantable medical device systems for treating ventricular tachyarrhythmias.

### BACKGROUND

Implantable cardiac rhythm management (CRM) systems, including pacemakers, implantable cardioverter/defibrillators (ICDs), and cardiac resynchronization therapy (CRT, CRT-D) devices have been used to deliver effective treatment to patients with serious cardiac arrhythmias. In particular, ICDs and CRT-D devices may treat cardiac tachyarrhythmias with a variety of tiered therapies ranging, for example, from delivering low energy pacing pulses timed to assist the heart in maintaining pumping efficiency to providing high-energy shocks to treat and/or terminate fibrillation. To effectively deliver these treatments, the CRM system must first identify the type of arrhythmia that is occurring, after which appropriate therapy may be delivered to the heart. In particular, it is desirable to avoid delivering high energy shocks in situations where a less aggressive form of therapy may be suitable. There is thus a need for improved systems and methods for arrhythmia identification and therapy discrimination.

### SUMMARY

The present invention, in one embodiment, is a method for controlling cardiac ventricular tachyarrhythmias using an implanted medical device. The method comprises the implanted medical device first acquiring a pressure signal representative of coronary venous pressure (CVP) from a pressure sensor implanted within a coronary vein of the patient, and deriving a CVP index based on the pressure signal. The implanted medical device also acquires a rate signal indicative of the patient's cardiac rate from an implanted rate sensor, and detects an onset of a ventricular tachyarrythmia (VT) episode based on the rate signal. The method further comprises monitoring the rate signal and the CVP index for an episode monitoring period. Responsive to the rate signal indicating a sustained tachycardia episode during the episode monitoring period, the implanted medical device selectively withholds anti-tachycardia therapy and extends the episode monitoring period based on the CVP index.

In another embodiment, the present invention is a method for controlling cardiac ventricular tachyarrhythmias using an implanted medical device, the method comprising the implanted medical device acquiring a pressure signal representative of CVP from a pressure sensor implanted within a coronary vein of the patient, and deriving a CVP index based on the pressure signal. The implanted medical device further acquires a rate signal indicative of the patient's cardiac rate from an implanted rate sensor, and detects an onset of a VT episode based on the rate signal. The method also comprises monitoring the rate signal and the CVP index for an episode monitoring period, and classifying the VT episode according to a degree of hemodynamic stability or hemodynamic instability based on the CVP index. Subsequently, the medical device delivers an anti-tachycardia therapy if the episode is classified as hemodynamically unstable, and withholds anti-tachycardia therapy and extends the episode monitoring period if the episode is classified as hemodynamically stable.

In yet another embodiment, the present invention is an implantable cardiac rhythm management system configured to perform the methods described above and below. In one embodiment, the system comprises a plurality of implantable medical electrical leads and an implantable pulse generator. The leads are configured to sense cardiac electrical activity and to deliver an electrical therapeutic stimulus generated by the pulse generator. At least one of the leads is configured for chronic implantation within a coronary vein of the patient's heart and includes a pressure sensor configured to generate a pressure signal indicative of fluid pressure within the coronary vein. The pulse generator is operatively coupled to the leads configured to generate an electrical therapeutic stimulus to a patient's cardiac tissue, and includes a control system configured to acquire the pressure signal and derive a CVP index based on the pressure signal and to acquire a rate signal indicative of the patient's cardiac rate from at least one of the implantable leads. The control system is further configured to detect an onset of a VT episode based on the rate signal, monitor the rate signal and the CVP index for an episode monitoring period, and classify the VT episode as hemodynamically stable or hemodynamically unstable based on the CVP index. In addition, the control system is configured to deliver an anti-tachycardia therapy if the episode is determined to be hemodynamically unstable, and to withhold anti-tachycardia therapy and extend the episode monitoring period if the episode is determined to be hemodynamically stable.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a schematic illustration of an implantable cardiac rhythm management (CRM) system according to one embodiment of the present invention in a deployed configuration.

FIG. 2 is a block diagram illustrating functional components of the implantable medical system of FIG. 1.

FIG. 3 is an illustration of coronary venous system pressure waveforms that can be obtained utilizing the CRM system of FIG. 1.

FIG. 4 is an illustration depicting a coronary venous pressure waveform and corresponding left ventricular pressure waveform during a ventricular tachyarrhythmia event.

FIG. 5 is a flow chart illustrating an exemplary method of treating a ventricular tachyarrhythmia using the CRM system of FIG. 1 according to one embodiment of the present invention.

FIG. 6 is a flow chart illustrating a method of treating a ventricular tachyarrhythmia using the CRM system of FIG. 1 according to another embodiment of the present invention.

FIG. 7 is a flow chart illustrating a method of treating a ventricular tachyarrhythmia using the CRM system of FIG. 1 in conjunction with the method of FIG. 6, according to another embodiment of the present invention.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

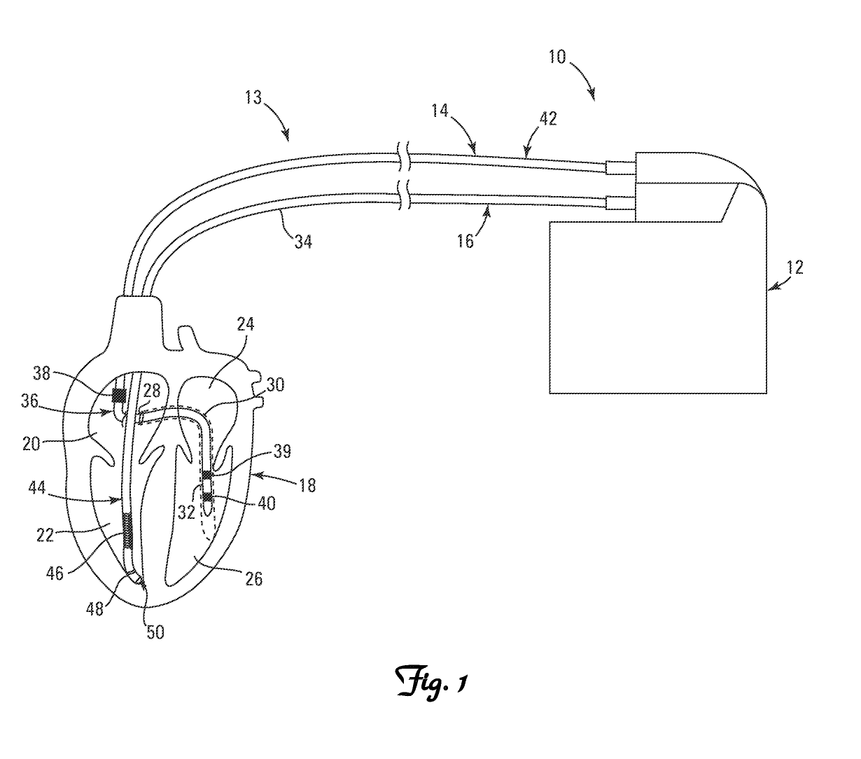

FIG. 1 is a schematic drawing of an implantable cardiac rhythm management (CRM) system 10 according to one embodiment of the present invention, shown in a deployed state. As shown in FIG. 1, the CRM system 10 includes a pulse generator 12 coupled to a cardiac lead system 13 including a pair of medical electrical leads 14, 16 deployed in a patient's heart 18, which includes a right atrium 20 and a right ventricle 22, a left atrium 24 and a left ventricle 26, a coronary sinus ostium 28 in the right atrium 20, a coronary sinus 30, and various coronary veins including an exemplary branch vessel 32 off of the coronary sinus 30. As discussed in detail below, the CRM system 10 is configured to treat cardiac arrhythmias, and in particular, ventricular tachyarrhythmia (VT) episodes, utilizing information regarding the patient's hemodynamic state for classifying the particular VT episodes for therapy discrimination.

As shown in FIG. 1, the lead 14 includes a proximal portion 42 and a distal portion 36, which as shown is guided through the right atrium 20, the coronary sinus ostium 28 and the coronary sinus 30, and into the branch vessel 32 of the coronary sinus 30. The distal portion 36 further includes pressure sensors 38, 39, and an electrode 40. As shown, the pressure sensor 39 and the electrode 40 are positioned on the lead 14 such that, when implanted, they are both located within the coronary branch vein 32. As further shown, the pressures sensor 38 is positioned on the lead 14 such that, when implanted, it is located within the right atrium 20. The illustrated position of the lead 14 may be used for delivering a pacing and/or defibrillation stimulus to the left side of the heart 18. Additionally, the lead 14 may also be partially deployed in other regions of the coronary venous system, such as in the great cardiac vein or other branch vessels for providing therapy to the left side or right side of the heart 18.

In the illustrated embodiment, the electrode 40 is a relatively small, low voltage electrode configured for sensing intrinsic cardiac electrical rhythms and/or delivering relatively low voltage pacing stimuli to the left ventricle 26 from within the branch coronary vein 32. In various embodiments, the lead 14 can include additional pace/sense electrodes for multi-polar pacing and/or for providing selective pacing site locations.

As further shown, in the illustrated embodiment, the lead 16 includes a proximal portion 34 and a distal portion 44 implanted in the right ventricle 22. In other embodiments, the CRM system 10 may include still additional leads, e.g., a lead implanted in the right atrium 20. The distal portion 44 further includes a flexible, high voltage electrode 46, a relatively low-voltage ring electrode 48, and a low voltage tip electrode 50 all implanted in the right ventricle 22 in the illustrated embodiment. The high voltage electrode 46 has a relatively large surface area compared to the ring electrode 48 and the tip electrode 50, and is thus configured for delivering relatively high voltage electrical stimulus to the cardiac tissue for defibrillation/cardioversion therapy, while the ring and tip electrodes 48, 50 are configured as relatively low voltage pace/sense electrodes. The electrodes 48, 50 provide the lead 16 with bi-polar pace/sense capabilities.

In various embodiments, the lead 16 includes additional defibrillation/cardioversion and/or additional pace/sense electrodes positioned along the lead 16 so as to provide multi-polar defibrillation/cardioversion capabilities. In one exemplary embodiment, the lead 16 includes a proximal high voltage electrode in addition to the electrode 46 positioned along the lead 16 such that it is located in the right atrium 20 (and/or superior vena cava) when implanted. Additional electrode configurations can be utilized with the lead 16. In short, any electrode configuration can be employed in the lead 16 without departing from the intended scope of the present invention.

In various embodiments, the lead 16 can be configured according to the various embodiment described in co-pending and commonly assigned U.S. Provisional Patent Application 61/088,270 titled “Implantable Lead and Coronary Venous Pressure Sensor Apparatus and Method” to Liu, et al. or commonly assigned U.S. Pat. No. 7,409,244 titled “Method and Apparatus for Adjusting Interventricular Delay Based on Ventricular Pressure,” to Salo, et al., the disclosures of which are incorporated herein by reference in their entireties. In other embodiments, the lead 16 with pressure sensor 39 and/or 38 can have other suitable configurations.

The pulse generator 12 is typically implanted subcutaneously within an implantation location or pocket in the patient's chest or abdomen. The pulse generator 12 may be any implantable medical device known in the art or later developed, for delivering an electrical therapeutic stimulus to the patient suitable for treating cardiac tachyarrhythmias. In various embodiments, the pulse generator 12 is an implantable cardioverter defibrillator (ICD) or a cardiac resynchronization (CRT) device configured for bi-ventricular pacing and including defibrillation capabilities (i.e., a CRT-D device). The pulse generator 12 includes hardware, software, and circuitry operable as a detection/energy delivery system configured to receive cardiac rhythm signals from the lead electrode(s) 40, 48, 50 and pressure signals from the pressure sensor(s) 38, 39, and also to deliver a therapeutic electrical stimulus to the electrodes 40, 48, 50.

In various embodiments, the CRM system 10 further includes an additional lead deployed in the right atrium 20, which lead may include one or more additional electrodes sensing intrinsic cardiac signals and/or delivering electrical stimuli to the cardiac tissue within the right atrium 20.

The pressure sensor 39 is operable to sense and to generate an electrical signal representative of a fluid pressure parameter within the coronary vein 32 in which it is implanted. The pressure sensor 39 can be any device, whether now known or later developed, suitable for sensing pressure parameters within the coronary venous system and generating and transmitting a signal indicative of such pressure parameters to another device, e.g., the pulse generator 12. In various embodiments, the pressure sensor 39 is configured to sense and generate a signal indicative of hydrostatic pressure within the coronary vein. In various embodiments, the pressure sensor 39 can be a micro-electrical-mechanical system (MEMS) device, which utilizes semiconductor techniques to build microscopic mechanical structures in a substrate made from silicon or similar materials. In various embodiments, the pressure sensor 39 can include a micro-machined capacitive or piezoresistive sensor exposed to the bloodstream. Other pressure sensor technologies, such as resistive strain gages, are known in the art and can also be employed as a pressure sensor 39.

In other exemplary embodiments, the pressure sensor 39 can include one or more piezoelectric elements. Such piezoelectric elements are configured to flex and/or deflect in response to changes in pressure within the coronary vein in which it is implanted, and to generate an output current or voltage proportional to the corresponding pressure change. In such embodiments, the pressure sensor 39 may advantageously be configured to sense fluid characteristics indicative of changes in coronary venous pressure during the cardiac cycle, e.g., dp/dt, systolic pressure, pulse pressure, cycle length which in turn can be monitored over time.

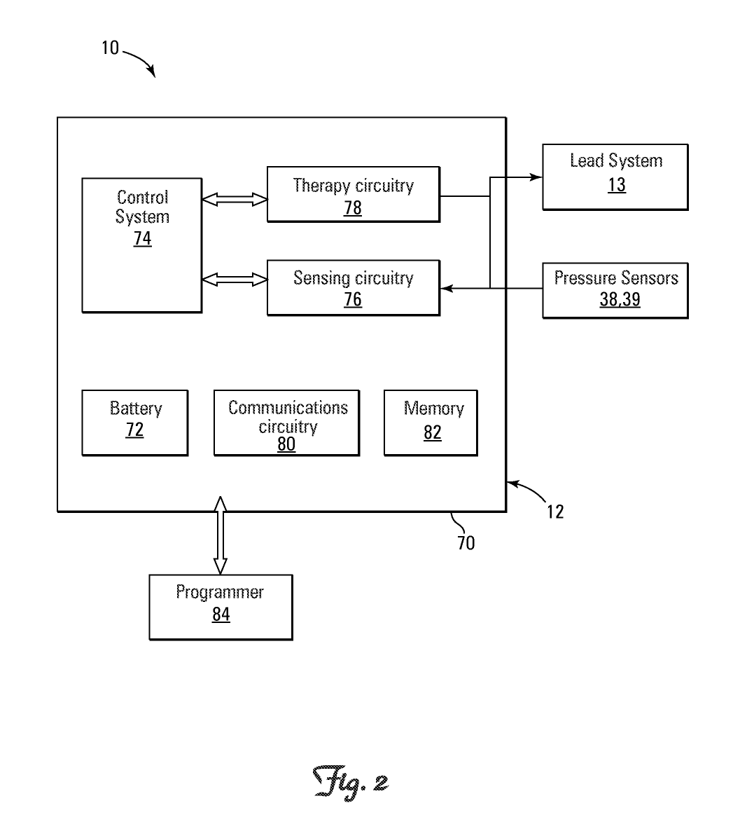

FIG. 2 is a schematic functional block diagram of an embodiment of the implantable medical system 10. As shown in FIG. 2, the system 10 is divided into functional blocks. The illustrated configuration is exemplary only, and there exist many possible configurations in which these functional blocks can be arranged. The example depicted in FIG. 2 is one possible functional arrangement. The system 10 includes circuitry for receiving cardiac electrical signals, coronary venous pressure signals, and in some embodiments, right atrial pressure signals from the heart 18 and generating and delivering electrical energy in the form of pace pulses or cardioversion/defibrillation pulses to the heart 18.

As discussed above, the cardiac lead system 13, which includes the leads 14, 16 may be implanted so that the cardiac electrodes 40, 48, 50 (see FIG. 1) contact heart tissue. The cardiac electrodes of the lead system 13 sense cardiac signals associated with electrical activity of the heart. In addition, the pressure sensors 38, 39 on the lead 14 detect and generate pressure signals indicative of blood pressure within the right atrium 20 and coronary vein 32, respectively. The sensed cardiac signals and pressure signals are transmitted to a the pulse generator 12 through the lead system 13. The cardiac electrodes and lead system 13 may be used to deliver electrical stimulation generated by the pulse generator 12 to the heart to mitigate various cardiac arrhythmias. The pulse generator 12, in combination with the cardiac electrodes and the lead system 13, may detect cardiac signals and deliver therapeutic electrical stimulation to any of the left and right ventricles and left and right atria, for example.

As shown, the pulse generator 12 includes circuitry encased in a hermetically sealed housing 70 suitable for implanting in a human body. Power is supplied by a battery 72 that is housed within the housing 70. In one embodiment, the pulse generator circuitry is a programmable microprocessor-based system, including a control system 74, sensing circuitry 76, therapy circuitry 78, communications circuitry 80, and memory 82. The memory 82 may be used, for example, to store programmed instructions for various pacing and defibrillation therapy and sensing modes, and also data associated with sensed cardiac signals or other physiologic data, e.g., blood pressure. The parameters and data stored in the memory 82 may be used on-board for various purposes and/or transmitted via telemetry to an external programmer unit 84 or other patient-external device, as desired. In various embodiments, the stored data can be uploaded by a clinician and/or transmitted over an advanced patient management (APM) system, such as the LATITUDE® system marketed by Boston Scientific Corporation.

The communications circuitry 80 allows the pulse generator 12 to communicate with the external programmer unit 84 and/or other patient-external system(s). In one embodiment, the communications circuitry 80 and the programmer unit 84 use a wire loop antenna and a radio frequency telemetric link to receive and transmit signals and data between the programmer 84 and communications circuitry 80 In this manner, programming commands may be transferred to the pulse generator 12 from the programmer 84 during and after implant. In addition, stored cardiac data may be transferred to the programmer unit 84 from the pulse generator 12, for example.

The sensing circuitry 76 detects cardiac signals sensed at the cardiac electrodes 40, 48, 50, as well as blood pressure signals generated by the pressure sensors 38, 39. The sensing circuitry 76 may include, for example, amplifiers, filters, ND converters and other signal processing circuitry. Cardiac signals and pressure signals processed by the sensing circuitry may be communicated to the control system 74.

The control system 74 is used to control various subsystems of the pulse generator 12, including the therapy circuitry 78 and the sensing circuitry 76. The control system 74 perform various functions, including, for example, arrhythmia analysis and therapy selection. An arrhythmia analysis section of the control system 74 may compare signals detected through the sensing circuitry 76 to detect or predict various cardiac arrhythmias, and to assist selection of appropriate therapies for the patient.

The therapy circuitry 78 is controlled by the control system 74 and may be used to deliver pacing stimulation pulses to the heart through one or more of the cardiac electrodes, according to a pre-established pacing regimen under appropriate conditions. Also, the therapy circuit 78 may deliver anti-tachycardia therapy such as relatively low-voltage anti-tachycardia pacing (ATP) pulses or relatively high-energy shocks to terminate or mitigate cardiac arrhythmias such as ventricular fibrillation detected or predicted by the control system 74.

As discussed above, certain pressure parameters associated with hemodynamic state of the heart can be utilized, e.g., by the control system 74 of the pulse generator 12, in an algorithm for VT identification and/or anti-tachyarrythmia therapy discrimination. In particular, selected pressure measurements can be utilized to determine whether a given VT episode is accompanied by hemodynamic stability or instability. In turn, this determination can be utilized to select an appropriate therapeutic response, which in some circumstances, may include withholding electrical stimulation and continuing an monitoring period. One such useful hemodynamic parameter is left ventricular (LV) pressure.

Table 1 below illustrates various exemplary cardiac pressure parameters during a VT episode which can be particularly useful in an algorithm for VT identification and/or anti-tachyarrythmia therapy discrimination as described in greater detail below. Specifically, three such pressure parameters that are of particular interest are left ventricular systolic pressure (LVsp), left ventricular pulse pressure (LVpp), and a maximum change in LV pressure over time (LVdp/dtmax) over a predetermined interval. Table 1 illustrates exemplary average values of LVsp, LVpp, and LVdp/dtmax (as a percentage of a baseline value for each respective parameter) obtained during a VT episode in an animal study.

TABLE 1

| Parameter | Stable VT | Unstable VT |
|---|---|---|
| LVsp | 37.46 | 31.21 |
| LVpp | 21.84 | 15.36 |
| LVdp/dtmax | 53.54 | 38.96 |

As shown in Table 1, relatively lower LVsp, LVpp, and LVdp/dtmax values are associated with a hemodynamically unstable ventricular tachyarrhythmia episode, whereas higher LVsp, LVpp, and LVdp/dtmax values can indicate a relatively hemodynamically stable episode. Thus, providing the control system 74 of the CRM system 10 with data indicative of LV pressure allows for VT episodes to be classified and binned according to the associated degree of hemodynamic stability (or instability) for the particular episodes. For example, the control system 74 can bin specific VT episodes as hemodynamically stable or hemodynamically unstable, and can select an appropriate therapy or withhold therapy altogether, depending on which bin the episode falls within.

Of course, the actual pressure parameter values listed in Table 1 are illustrative only. Additionally, the actual threshold pressure parameter values for characterizing hemodynamically stable and unstable VT episodes are not universal, but rather can be selected or programmed by the clinician based on the patient's particular clinical history and needs.

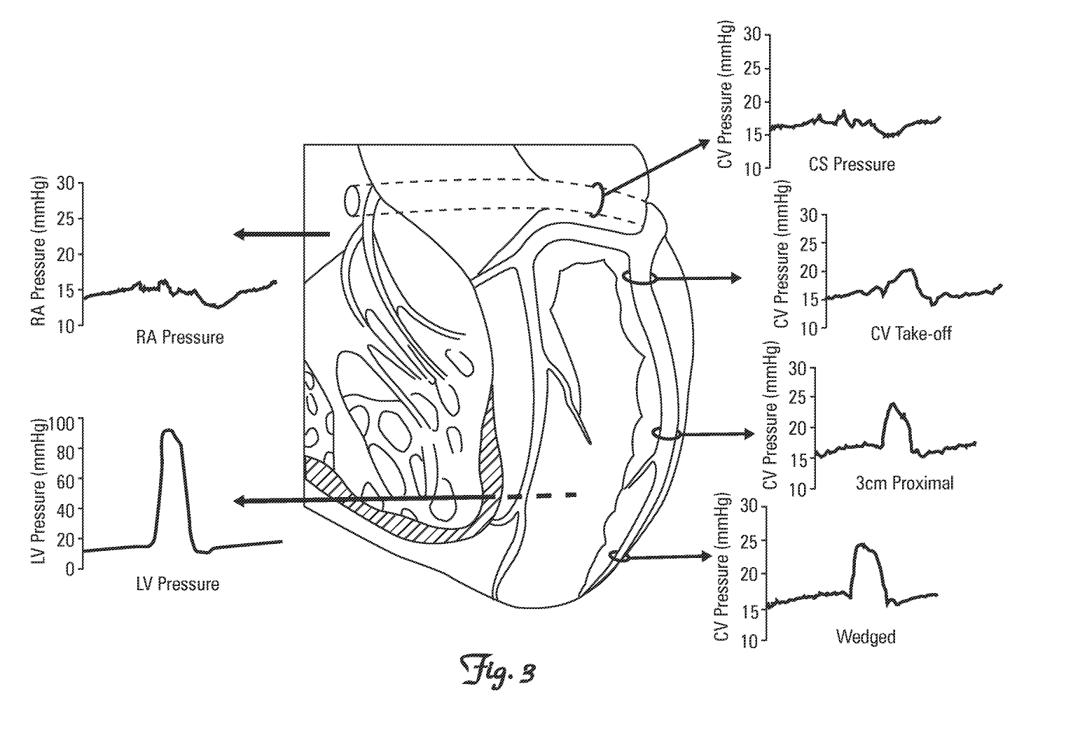

As explained above, the pressure sensors 38, 39 are configured to detect and generate pressure signals representative of fluid pressure within the right atrium 20 and the coronary vein 32, respectively. From these pressure signals, pressure waveforms can be derived and evaluated by the sensing circuitry 76 and the control system of the pulse generator 12. FIG. 3 illustrates pressure waveforms obtained from the right atrium (RA), left ventricle (LV), coronary sinus (CS) and various locations in a coronary vein (CV) in another exemplary animal study. As shown, the coronary venous pressure (CVP) waveform takes on the same general shape as the LV waveform, particularly where the CVP is taken from a location lower in the coronary vein (as indicated by the “Wedged” pressure reading).

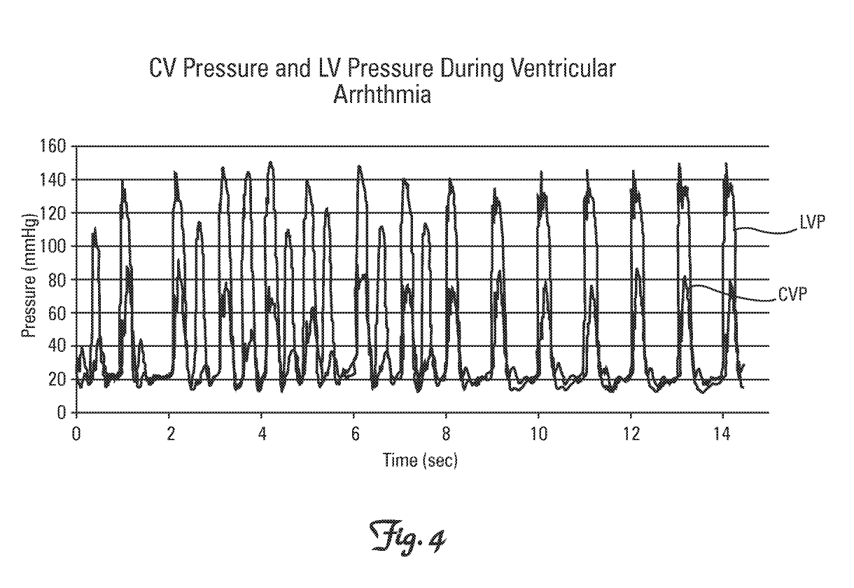

FIG. 4 is an illustration depicting a CVP waveform and corresponding LVP waveform during a VT episode in an exemplary animal study. As can be seen in FIG. 4, the CVP and LVP waveforms continue to correlate closely to one another during the depicted VT episode. Thus, in view of the close correlation between coronary venous pressure and LV pressure, VT classification/binning can be accomplished using CVP data to the same extent as can be accomplished based on LVP parameters as described above. Thus, the CRM system 10 provides the capability for chronically monitoring hemodynamic stability associated with VT episodes, which in turn enables tachycardia therapy discrimination based at least in part on the degree of hemodynamic stability associated with the episodes.

Additionally, in various embodiments, right atrial pressure information, e.g., obtained from the pressure sensor 38 located in the right atrium 20, can be utilized by the control system 74 in conjunction with the CVP information to assist in arrhythmia identification (e.g., to identify super ventricular tachycardia or ventricular tachycardia) and/or to guide the therapy selection in accordance with the methods described below. In the illustrated embodiments, the right atrial pressure is obtained directly from the pressure sensor 38 located in the right atrium 20, while in other embodiments, right atrial pressure may be sensed using one or more pressure sensors located in the coronary sinus 30.

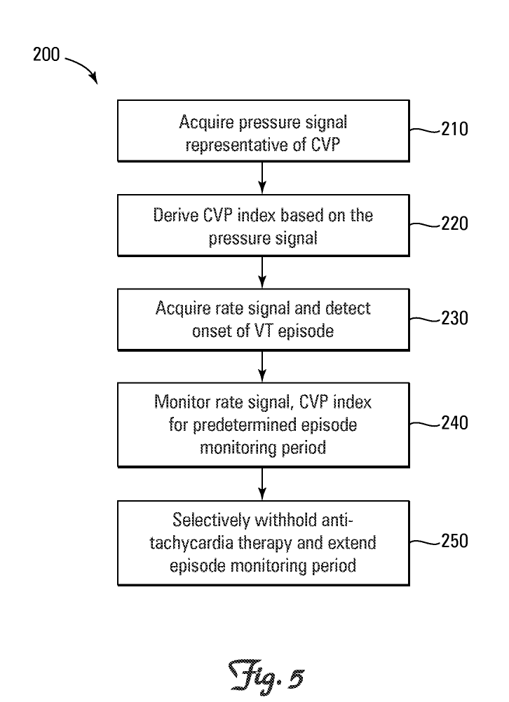

FIG. 5 is a flow chart illustrating an exemplary method 200 utilizing both cardiac rate signal data and coronary venous pressure signal data in arrhythmia identification and therapy selection/discrimination. The method 200 can be carried out by the CRM system 10 shown and described above. As shown in FIG. 5, the method 200 begins with the CRM system 10 acquiring a pressure signal representative of CVP from a pressure sensor (e.g., the pressure sensor 39 of FIG. 1) implanted within a coronary vein of the patient (block 210). The CRM system then derives an appropriate CVP index based on the pressure signal (block 220). The CVP index can be any appropriate index derived from the pressure signal that is useful in assessing the hemodynamic stability associated with a VT episode. In some embodiments, the pressure sensor 38 disposed in the right atrium 20 may also be utilized to derive a pressure index for use by the control system 74.

For example, in one embodiment, the control system 74 may be configured to bin the VT episodes based on CVpp (which as explained above, correlates closely to LVpp) during the episode. Accordingly, in this embodiment, the control system 74 may be programmed to calculate an average CVpp value over a predetermined interval, e.g., a predetermined number of beats/cycles, or a predetermined time period, during the VT episode. Similar calculations can be made using other selected CVP indexes, e.g., CVsp or CVdp/dt. Additionally, the control system 74 can be configured to calculate and monitor more than one CVP index at a time.

As further shown, the method 200 also includes acquiring a rate signal indicative of the patient's cardiac rate from an implanted rate sensor, and detecting the onset of a VT episode based on the rate signal (block 230). Various rate signals suitable for assessing and detecting VTs are well known and need not be discussed in detail here.

Upon detection of the onset of the VT episode, the rate signal and CVP index are monitored for a predetermined episode monitoring period (block 240). In general, the episode monitoring period is a predetermined time interval programmed by the clinician. However, it is contemplated that, in one embodiment, the episode monitoring period can be adjusted by the control system 74 over time based on the particular patient's history.

Subsequently, in response to the rate signal indicating a sustained tachycardia episode at the termination of the episode monitoring period and based on the CVP index, the method 200 includes selectively withholding anti-tachycardia therapy and extending the episode monitoring period (block 250). In one embodiment, for example, the control system 74 analyzes the calculated average CVP index value during the episode and compares this average value to a baseline CVP index value obtained prior to the onset of the VT episode. Based on this analysis, the hemodynamic stability of the patient during the VT episode is determined by the control system 74 using criteria such as described above in connection with Table 1.

Thus, in one embodiment, the control system 74 determines whether the VT episode is hemodynamically stable or hemodynamically unstable, and the control system 74 bases its therapy decision in this determination. In particular, where the episode is determined to be hemodynamically stable, the control system 74 withholds delivery of anti-tachycardia therapy and extends the episode monitoring period. In other words, the CRM system 10 is configured to defer delivery of anti-tachycardia therapy in response to a sustained VT episode in situations where the hemodynamic data obtained from the CRM system 10 indicates that the patient's hemodynamic status is not materially compromised. In this way, the likelihood that the CRM system 10 will unnecessarily delivering anti-tachycardia therapy, and in particular, a potentially painful high energy shock, is advantageously reduced.

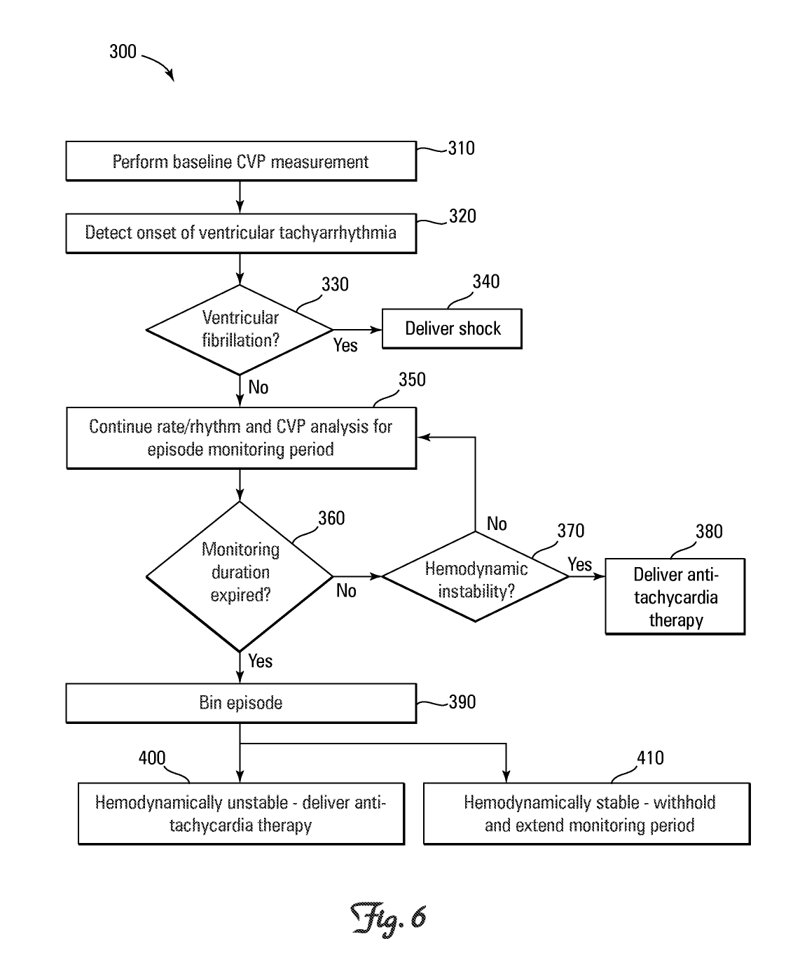

FIG. 6 is a flow chart illustrating a method 300 of treating a ventricular tachyarrhythmia using the CRM system 10 according to one embodiment of the present invention. As shown in FIG. 6, initially, CVP is sensed and analyzed to establish a baseline CVP index value (block 310). The CVP index can be based on any suitable CVP parameter (e.g., CVpp, CVsp) for assessing the patient's hemodynamic state during a VT episode, as discussed above in connection with the method 200. The baseline CVP index value is stored in the memory 82 of the CRM system 10 (see FIG. 2). In this way, the baseline CVP index value can be uploaded by a clinician and/or transmitted, along with other useful data, over an advanced patient management (APM) system, such as the LATITUDE® system marketed by Boston Scientific Corporation.

Next, as shown in FIG. 6, the onset of a VT episode is detected by the CRM system 10 (block 320) using known techniques. After detecting the onset of the VT episode, in the illustrated embodiment, the control system 74 of the CRM system 10 determines whether the episode is a ventricular fibrillation episode (block 330). If so, the CRM system 10 delivers a ventricular fibrillation therapy, e.g., a high-energy defibrillation shock, as is known in the art (block 340).

If, however, the CRM system 10 determines at block 330 that the episode is not a ventricular fibrillation episode, the system 10 initiates cardiac rate signal and CVP analysis for an episode monitoring period (block 350). As discussed above, in various embodiments, the episode monitoring period may have a predetermined and preprogrammed duration.

As further shown in FIG. 6, the control system 74 also tracks the duration of the episode monitoring period (block 360). During the episode monitoring period (i.e., the where the duration has not expired), if the control system 74 identifies hemodynamic instability, e.g., as characterized by significantly compromised hemodynamic function based on the CVP index (block 370), the control system 74 will direct the therapy circuitry 78 to deliver an appropriate anti-tachycardia therapy, e.g., a defibrillation shock or anti-tachycardia pacing (block 380).

Upon the expiration of the episode monitoring period, if the VT episode is a sustained episode, the control system 74 classifies or bins the episode according to the hemodynamic stability of the patient during the episode (block 390). This binning is done by the control system 74 based on its analysis of the rate signal analysis and the CVP index. In one embodiment, for example, the control system 74 analyzes the calculated average CVP index value during the episode and compares this average value to a baseline CVP index value obtained prior to the onset of the VT episode. Based on this analysis, the VT episode is classified according to its hemodynamic stability, as determined by the control system 74 using criteria such as described above in connection with Table 1. For example, if the selected CVP index (e.g., average CVpp over a predetermined interval), measured as a percentage of the baseline average CVpp, is equal to or greater than the predetermined threshold value, the control system 74 classifies the episode as hemodynamically stable. In contrast, if this measured CVpp value is below a predetermined threshold, the control system will classify the episode as hemodynamically unstable.

Upon classifying the VT episode, the control system 74 may then apply an appropriate therapy protocol based on the hemodynamic stability of the episode. As shown in FIG. 6, if the episode is classified as hemodynamically unstable, the control system 74 will direct the therapy circuitry 78 (see FIG. 2) to deliver an anti-tachycardia therapy according to the program established by the clinician (block 400). As further shown in FIG. 6, if the episode is classified as hemodynamically stable, rather than deliver an anti-tachycardia therapy, the control system 74 will withhold therapy and will extend the episode monitoring period (block 410). The control system 74 will then continue to monitor the cardiac rate signal and the CVP index value during this extended monitoring period. The CRM system 10 can then repeat the monitoring and classification steps as appropriate.

While FIG. 6 illustrates, in essence, only two episode bins—i.e., hemodynamically unstable and hemodynamically stable—in various embodiments, a wide range of bins can be defined to provide a wide range of therapy decisions. For example, under the general classification of hemodynamically unstable VTs, additional bins can be defined based on the degree of hemodynamic instability indicated by the CVP index analysis. In turn, the aggressiveness of the anti-tachycardia therapy will also depend on which bin the particular episode falls within. Thus, if the CVP index indicates only moderate instability, a relatively less aggressive therapy, such as anti-tachycardia pacing, may be employed. However, if the episode is classified as relatively highly unstable, a more aggressive therapy will be applied, e.g., a high-energy shock. Of course, the cardiac rate signal can also be considered by the control system 74 in classifying the particular episode.

Additionally, in one embodiment, the control system 74 can be programmed to adjust the duration of the extended episode monitoring period based on the CVP index value even where the episode is classified as hemodynamically stable. For example, where the CVP index value falls near the stable/unstable transition threshold, a relatively short duration will be assigned to the extended episode monitoring period.

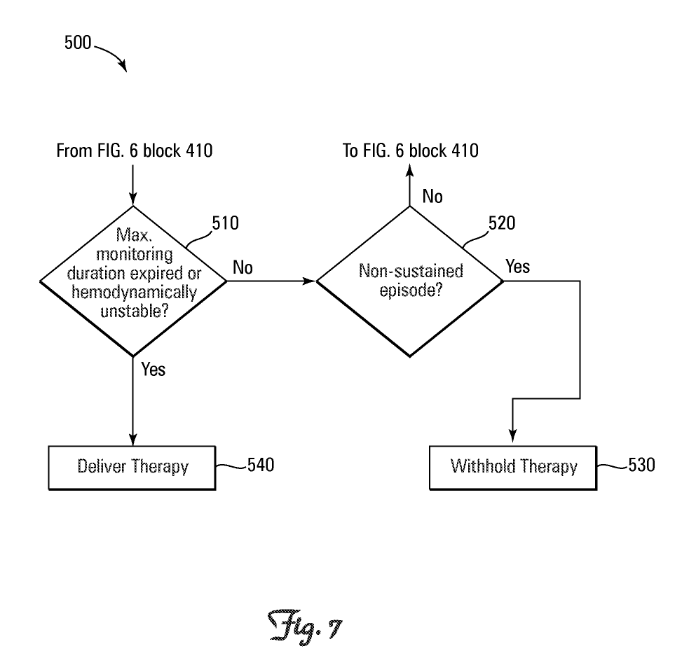

FIG. 7 illustrates a method 500 of treating a ventricular tachyarrhythmia using the CRM system 10 in conjunction with the method 300 depicted in FIG. 6, according to another embodiment of the present invention. As shown in FIG. 7, where the control system 74 has withheld therapy and extended the episode monitoring period based on classifying the episode as hemodynamically stable, the control system 74 will then determine whether a maximum episode monitoring period duration has expired (block 510). The maximum episode monitoring duration can represent the longest duration in which the episode will be monitored without applying an anti-tachycardia therapy. The maximum episode monitoring duration may, in various embodiments, be determined by the clinician and programmed into the control system 74. Additionally, or alternatively, the control system 74 may be programmed to vary the maximum episode monitoring duration in accordance with the episode classification scheme discussed above. That is, where the CVP index value falls near the stable/unstable transition threshold, the maximum episode monitoring period may be selected to be relatively short. In turn, where the CVP index indicates that the episode is, relatively speaking, highly stable, a longer maximum episode monitoring duration may be selected by the control system 74 according to its programmed instructions.

As further shown in FIG. 7, if the maximum episode monitoring duration has not expired, the control system determines, based on the rate signal, if the episode is a non-sustained episode (block 520). If the episode is non-sustained, the control system will withhold delivering therapy because the VT episode has concluded (block 530). If, however, the episode continues, therapy will be withheld and the control system will continue to monitor the rate signal and CVP index as shown in block 390 of FIG. 6.

As also shown in FIG. 7, where the maximum episode monitoring duration has expired, or if the episode is classified as hemodynamically unstable during the extended monitoring period, the control system will direct the therapy circuitry 78 to deliver an anti-tachycardia therapy (block 540). Here again, as explained above, the aggressiveness of the delivered therapy can be varied based on the degree of hemodynamic instability associated with the particular episode.

Embodiments of methods and devices in accordance with the present invention may be implemented to adapt to changing patient conditions, medications, and/or cardiac pathology over time. Adaptation over time may possibly decrease the time to effective therapy, and increases the ratio of pain-free to painful therapy, by automatically incorporating information about that patient's prior therapy efficacy when deciding what type of therapy, if any, should be delivered to the current tachyarrhythmia episode.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A method for controlling cardiac ventricular tachyarrhythmias using an implanted medical device, the method comprising the implanted medical device:
acquiring a pressure signal representative of coronary venous pressure (CVP) from a pressure sensor implanted within a coronary vein of the patient, and deriving a CVP index based on the pressure signal;
acquiring a rate signal indicative of the patient's cardiac rate from an implanted rate sensor;
detecting an onset of a ventricular tachyarrythmia (VT) episode based on the rate signal;
monitoring the rate signal and the CVP index for an episode monitoring period; and
responsive to the rate signal indicating a sustained tachycardia episode during the episode monitoring period, selectively withholding anti-tachycardia therapy and extending the episode monitoring period based on the CVP index.

2. The method of claim 1 wherein withholding anti-tachycardia therapy and extending the episode monitoring period includes withholding anti-tachycardia therapy and extending the episode monitoring period in response to the CVP index indicating a hemodynamically stable VT episode.

3. The method of claim 1 further comprising selectively delivering anti-tachycardia therapy or withholding anti-tachycardia therapy based on the CVP index.

4. The method of claim 3 wherein selectively applying the anti-tachycardia therapy includes applying an anti-tachycardia therapy in response to the CVP index indicating a moderately hemodynamically unstable VT episode.

5. The method of claim 1 further comprising applying a defibrillation shock during the episode monitoring period in response to the CVP index indicating a hemodynamically unstable VT episode.

6. The method of claim 1 further comprising, responsive to the rate signal indicating a sustained tachycardia episode, applying anti-tachycardia therapy when a duration of the episode monitoring period equals or exceeds a predetermined maximum episode duration.

7. The method of claim 1 wherein the CVP index is a relative change in a sensed average CVP relative to a baseline average CVP.

8. The method of claim 1 wherein the anti-tachycardia therapy is an anti-tachycardia pacing therapy.

9. The method of claim 1 wherein the anti-tachycardia therapy is a defibrillation shock.

10. A method for controlling cardiac ventricular tachyarrhythmias using an implanted medical device, the method comprising the implanted medical device:
acquiring a pressure signal representative of CVP from a pressure sensor implanted within a coronary vein of the patient, and deriving a CVP index based on the pressure signal;
acquiring a rate signal indicative of the patient's cardiac rate from an implanted rate sensor;
detecting an onset of a VT episode based on the rate signal;
monitoring the rate signal and the CVP index for an episode monitoring period;
classifying the VT episode according to a degree of hemodynamic stability or hemodynamic instability based on the CVP index;
delivering an anti-tachycardia therapy if the episode is classified as hemodynamically unstable; and
withholding anti-tachycardia therapy and extending the episode monitoring period if the episode is classified as hemodynamically stable.

11. The method of claim 10 wherein classifying the VT episode is performed upon expiration of the episode monitoring period in response to the VT episode being a sustained episode.

12. The method of claim 10 wherein classifying the VT episode includes classifying the VT episode as hemodynamically stable, moderately hemodynamically unstable, or highly hemodynamically unstable.

13. The method of claim 10 wherein classifying the VT episode includes classifying the VT episode as being associated with a first or second degree of hemodynamic instability.

14. The method of claim 13 wherein delivering the anti-tachycardia therapy if the episode is classified as hemodynamically unstable includes applying a first anti-tachycardia therapy if the VT episode is classified as being associated with the first degree of hemodynamic instability, and applying a second anti-tachycardia therapy if the VT episode is classified as being associated with the second degree of hemodynamic instability.

15. The method of claim 10 further comprising applying a defibrillation shock during the episode monitoring period in response to the CVP index indicating a hemodynamically unstable VT episode.

16. The method of claim 10 further comprising, responsive to the rate signal indicating a sustained tachycardia episode, applying anti-tachycardia therapy when a duration of the episode monitoring period equals or exceeds or equals a predetermined maximum episode duration.

17. The method of claim 16 wherein the predetermined maximum episode duration is selected based on the CVP index.

18. An implantable cardiac rhythm management system comprising:
a plurality of implantable medical electrical leads configured to sense cardiac electrical activity and to deliver an electrical therapeutic stimulus to cardiac tissue, at least one of the leads being configured for chronic implantation within a coronary vein of the patient's heart and including a pressure sensor configured to generate a pressure signal indicative of fluid pressure within the coronary vein;
an implantable pulse generator operatively coupled to the leads configured to generate the electrical therapeutic stimulus, the pulse generator including a control system configured to:
acquire the pressure signal and derive a CVP index based on the pressure signal;
acquire a rate signal indicative of the patient's cardiac rate from at least one of the implantable leads;
detect an onset of a VT episode based on the rate signal;
monitor the rate signal and the CVP index for an episode monitoring period; and
classify the VT episode as hemodynamically stable or hemodynamically unstable based on the CVP index;
deliver an anti-tachycardia therapy if the episode is determined to be hemodynamically unstable; and
withhold anti-tachycardia therapy and extending the episode monitoring period if the episode is determined to be hemodynamically stable.

19. The system of claim 18 wherein the control system is further configured to assign a maximum duration to the episode monitoring period based on the CVP index.

20. The system of claim 18 wherein the control system is configured to select the anti-tachycardia therapy based on a degree of hemodynamic instability associated with the VT episode as indicated by the CVP index.
